(19) United States
(12) Patent Application Publication
(10) Pub. No.: US 2025/0270205 A1
(43) Pub. Date: Aug. 28, 2025

(54) COMPOSITIONS AND METHODS FOR INHIBITING TREM-1

(71) Applicant: AUGUSTA UNIVERSITY RESEARCH INSTITUTE, INC., Augusta, GA (US)

(72) Inventors: Anatoli HORUZSKO, Evans, GA (US); Iryna LEBEDYEVA, Augusta, GA (US); Thomas ALBERS, Troy, AL (US); Ashwin AJITH, Augusta, GA (US)

(73) Assignee: AUGUSTA UNIVERSITY RESEARCH INSTITUTE, INC., Augusta, GA (US)

(21) Appl. No.: 18/245,217

(22) PCT Filed: Sep. 20, 2021
(86) PCT No.: PCT/US2021/051072

§ 371 Date: Mar. 14, 2023

Related U.S. Application Data
(60) Provisional application No. 63/080,708, filed on Sep. 19, 2020.

(51) Int. Cl. C07D 471/04; A61K 31/439; A61P 35/00

(52) U.S. Cl. CPC C07D 471/04; A61K 31/439; A61P 35/00

(57) ABSTRACT

Provided herein are compounds of formula I or an enantiomer, solvate, or a pharmaceutically acceptable salt thereof. Also provided are pharmaceutical compositions and medicaments that include the compounds described herein as well as methods of treating inflammatory disease, cardiovascular disease, and cancer.

### STATEMENT REGARDING FEDERALLY SPONSORED RESEARCH

This invention was made with government support under RO1 CA172230 awarded by the National Institutes of Health. The government has certain rights in the invention.

### TECHNICAL FIELD OF THE INVENTION

Aspects of the invention are generally directed to small molecule inhibitors of the triggering receptor expressed on myeloid cells 1 (TREM-1) and methods of use thereof.

### BACKGROUND OF THE INVENTION

The inflammatory response is an extensive network in which innate immune cells play a major role. The Triggering receptor expressed on myeloid cell 1 (TREM-1) plays a key role in the amplification and signaling of the inflammatory response. TREM-1 is highly expressed by neutrophils, monocytes, and macrophages. Activation of TREM-1 causes increased secretion of proinflammatory cytokines and chemokines, such as Interleukin-8 (IL-8), IL-1b, IL-6, CCL2, CCL9, CXCL2. Further, published data show that TREM-1 plays a role in malignancies and chronic inflammatory diseases that may serveto predispose a subject to cancer. TREM-1 also plays a role in the pathogenesis of acute and chronic cardiovascular conditions, and is a crucial mediator of septic shock, especially, in cytokine release syndrome.

Therapeutic use of blockers specific for TREM-1 is still limited to pre-clinical animal models. TREM-1 blockers include peptides, TREM-1 Fc fusion protein, small molecules, and monoclonal antibodies. Because peptide therapeutics have a limited lifespan due to degradation, it is more efficient to develop the TREM-1-specific small molecule inhibitors. Recently it has been published that morin hydrate (MH; 2′,3,4′,5,7-pentahydroxyflavone), a member of the flavonoids, inhibits TREM-1/TLR4-mediated inflammatory response in murine macrophages and protects against acute liver injury. Because targeting both receptors, TREM-1 and TLR-4, is not advantageous in the most clinical pathological conditions, the development of TREM-1-specific small molecule inhibitors is an attractive strategy to diminish acute and chronic inflammation.

Therefore, it is an object of the invention to provide a TREM-1 specific antagonist and, specifically, small molecule TREM-1 antagonists.

It is another object of the invention to provide pharmaceutical compositions containing small molecule TREM-1 antagonists that specifically inhibit TREM-1.

### SUMMARY OF THE INVENTION

TREM-1 small molecule inhibitors or antagonist compositions and methods of their use are provided.

In one aspect, the invention provides a compound of Formula I:

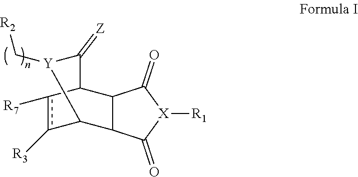

wherein=
[symbol] is a single or a double bond;
X and Y are each independently N or CH;
Z is independently CH₂, O, S, NH;
R₁ is independently alkyl, alkenyl, alkoxy, cycloalkyl, aryl, heteroaryl, wherein each of which is unsubstituted or substituted with H, OH, halogen, —COOH, —COOR₄, CH₂OH, —CH₂OR₄, —COSR₄, —CONH₂, —CH₂NH₂, CH₂NHR₅, —CH₂N₅R₆, —NCO, —CH₂-halogen, —CHO, —CN, —CONH—CO—R₄, —CH₂O—CO—O—R₄; NO₂, NH₂, NHR₅, NR₅R₆;
R₂ is independently alkyl, alkenyl, alkoxy, cycloalkyl, aryl, heteroaryl, wherein each of which is unsubstituted or substituted with H, OH, halogen, —COOH, —COOR₄, CH₂OH, —CH₂OR₄, —COSR₄, —CONH₂, —CH₂NH₂, CH₂NHR₅, —CH₂N₅R₆, —NCO, —CH₂-halogen, —CHO, —CN, —CONH—CO—R₄, —CH₂O—CO—O—R₄; NO₂, NH₂, NHR₅, NR₅R₆;
R₃ is independently H, OH, halogen, —COOH, —COOR₄, CH₂OH, —CH₂OR₄, —COSR₄, —CONH₂, —CH₂NH₂, CH₂NHR₅, —CH₂N₅R₆, —NCO, —CH₂-halogen, —CHO, —CN, —CONH—CO—R₄, —CH₂O—CO—O—R₄; NO₂, NH₂, NHR₅, NR₅R₆; and
R₄ is independently H, halogen, alkyl, aryl, heteroaryl;
R₅ and R₆ are independently H, halogen, alkyl, aryl, heteroaryl;
R₇ is independently H, CN, halogen, alkyl, alkoxy, aryl, heteroaryl; and
n is 1, 2, 3, 4, 5 or 6;
or an enantiomer, tautomer, isomer, exo and endo stereoisomers, bioisosteres, hydrate, solvate, racemate, deuterated analogs, zwitterion, polymorph, prodrug, or a pharmaceutically acceptable salt thereof.

In another aspect, the invention provides a compound of Formula II:

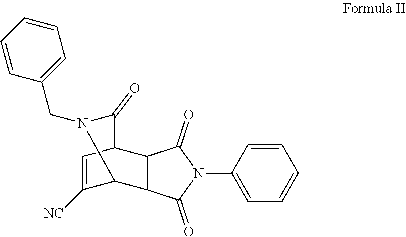 [SMILES: N#CC1=CC2C(=O)N(Cc3ccccc3)C1C1C(=O)N(c3ccccc3)C(=O)C21]

or an enantiomer, tautomer, isomer, exo and endo stereoisomers, bioisosteres, hydrate, solvate, racemate, deuterated analogs, zwitterion, polymorph, prodrug, or a pharmaceutically acceptable salt thereof.

In other aspects, the invention provides a process for preparing a compound of formula I.

In another aspect, the invention provides a pharmaceutical composition comprising a compound or a pharmaceutically acceptable salt thereof, and a pharmaceutically acceptable carrier, adjuvant or vehicle.

In some aspects, the invention provides a method of inhibiting TREM-1, the method comprising contacting the TREM-1 cells with a compound or a pharmaceutical composition thereof.

In other aspects, the invention provides a method of treating a TREM-related disease, disorder, or condition in a subject in need thereof comprising administering to the subject a therapeutically effective amount of the compound or a pharmaceutical composition thereof.

In a further aspect, the TREM-related disease, disorder, or condition is inflammatory disease, cardiovascular disease, and cancer.

In yet a further aspect, the invention provides a method of treating an inflammatory disease, disorder, or condition in a subject in need thereof comprising administering to the subject a therapeutically effective amount of the compound or pharmaceutical composition thereof.

In another aspect, the invention provides a method of treating a cardiovascular disease, disorder, or condition in a subject in need thereof comprising administering to the subject a therapeutically effective amount of the compound or a pharmaceutical composition thereof.

In yet a further aspect, the invention provides a method of treating a cancer in a subject in need thereof comprising administering to the subject a therapeutically effective amount of the compound or a pharmaceutical composition thereof.

### BRIEF DESCRIPTION OF THE DRAWINGS

FIGS. 1A-1D show computer generated graphical images of receptor-based virtual screening of TREM-1 inhibitors from the NCI Diversity Database. FIG. 1A shows NCI 37553 binding. FIG. 1B shows NCI 80313 binding. FIG. 1C shows NCI 118818 binding. FIG. 1D shows NCI 601359 binding.

FIG. 2A is a diagram showing that the engagement of human TREM-1 receptor activates a reporter cell line. FIG. 2B is a scatter plot showing that engagement of TREM-1 receptors on reporter cells with specific agonist antibody AF1278 (R&D Systems) resulted in the expression of green fluorescent protein (GFP) determined by flow cytometry. FIG. 2C is a line graph of the flow cytometry data of FIG. 2B.

FIG. 3A shows chemical structures of compound 118818 and compound VJDT8293, a highly efficient TREM-1 inhibitor with minimal cytotoxicity. FIG. 3B shows inhibitory effects of Morin Hydrate (MH), NCI 118818, and VIDT 8293 compounds on TREM-1-mediated production of human proinflammatory cytokines by activated neutrophils. FIG. 3C shows VJDT 8293 compound provides a much stronger inhibitory effect on TREM-1 mediated proinflammatory cytokine production by activated human neutrophils with less cytotoxicity.

FIG. 4 shows VJDT 8293 treatment significantly attenuates inflammatory cell infiltration at early stages of liver fibrosis. FIG. 4a shows control group receiving DMSO vehicle showed significant infiltration of macrophages (10-15%) and inflammatory neutrophils (15-22%) producing IL-1B, TNF, and TGF-β1 due to CCl4 injury. In contrast, FIG. 4b shows VJDT treatment had strong protective effect to liver injury exhibiting significantly reduced infiltration of both populations: macrophages (3-6%, p<0.001), and neutrophils (8-10%, p<0.01). Moreover, VJDT treatment additionally significantly decreased cytokine secretion in neutrophils in comparison to the control group. Control mice with no CCl4 injury showed minimal level of infiltrating cells and a normal population of resident Kupffer cells (10-15%).

FIG. 5 shows VJDT 8293 treatment inhibits tumor cell proliferation, migration, and plasticity. FIG. 5A shows VJDT treatment significantly inhibits B16F10 murine melanoma cell migration in a wound healing assay. FIG. 5B shows VIDT treatment significantly inhibites cell proliferation. FIG. 5C shows VIDT treatment produces significant cell cycle arrest in the S and G2 phases of cellular division.

FIG. 6 shows IC₅₀ of VIDT 8293 as 14.65 μM inhibits cell proliferation of human hepatocellular carcinoma cells (HepG2) in vitro after 72 hrs treatment.

### DETAILED DESCRIPTION OF THE INVENTION

### I. Definitions

The following terms are intended to have the meanings presented therewith below and are useful in understanding the description and intended scope of the present invention.

When describing the invention, which may include compounds, pharmaceutical compositions containing such compounds, and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term ‘substituted’ is to be defined as set out below. It should be further understood that the terms “groups” and “radicals” can be considered interchangeable when used herein.

The articles “a” and “an” may be used herein to refer to one or to more than one (i.e., at least one) of the grammatical objects of the article. By way of example “an analogue” means one analogue or more than one analogue.

As used herein, the term “pharmaceutical composition” means a mixture comprising a pharmaceutically acceptable active ingredient, in combination with suitable pharmaceutically acceptable excipients. In one embodiment the pharmaceutically acceptable ingredient is a pharmaceutically acceptable acid addition salt of the compound of formula I and II, or a solvate or hydrate of this acid addition salt.

Pharmaceutical excipients are substances other than the pharmaceutically acceptable active ingredient which have been appropriately evaluated for safety and which are intentionally included in an oral solid dosage form. For example, excipients can aid in the processing of the drug delivery system during its manufacture, protect, support or enhance stability, bioavailability or patient acceptability, assist in product identification, or enhance any other attribute of the overall safety, effectiveness or delivery of the drug during storage or use. Examples of excipients include, for example but without limitation inert solid diluents (bulking agent e.g., lactose), binders (e.g., starch), glidants (e.g., colloidal silica), lubricants (e.g., non-ionic lubricants such as vegetable oils), disintegrants (e.g., starch, polivinylpyrrolidone), coating better polymers (e.g., hydroxypropyl methylcellulose), colorants (e.g., iron oxide), and/or surfactants (e.g., non-ionic surfactants).

As used herein, the term “pharmaceutical formulation” means a composition in which different chemical substances, including the active drug, are combined to produce a final medicinal product. Examples of formulation include enteral formulations (tablets, capsules), parenteral formulations (liquids, lyophilized powders), or topical formulations (cutaneous, inhalable).

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a VJDT8293 or derivatives thereof that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. More particularly, such salts are formed with hydrobromic acid, hydrochloric acid, sulfuric acid, toluenesulfonic acid, benzenesulfonic acid, oxalic acid, maleic acid, naphthalene-2-sulfonic acid, naphthalene-1,5-disulfonic acid, 1-2-ethane disulfonic acid, methanesulfonic acid, 2-hydroxy ethanesulfonic acid, phosphoric acid, ethane sulfonic acid, malonic acid, 2-5-dihydroxybenzoic acid, or L-Tartaric acid.

The term “pharmaceutically acceptable cation” refers to an acceptable cationic counter-ion of an acidic functional group. Such cations are exemplified by sodium, potassium, calcium, magnesium, ammonium, tetraalkylammonium cations, and the like.

“Pharmaceutically acceptable vehicle” refers to a diluent, adjuvant, excipient or carrier with which a compound of the invention is administered.

“Solvate” refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

The terms “inert solid diluent” or “solid diluent” or “diluents” refer to materials used to produce appropriate dosage form size, performance and processing properties for tablets and/or capsules. An inert solid diluent can be also referred to as filler or filler material. Particular examples of diluents include cellulose powdered, silicified microcrystalline cellulose acetate, compressible sugar, confectioner's sugar, corn starch and pregelatinized starch, dextrates, dextrin, dextrose, erythritol, ethylcellulose, fructose, fumaric acid, glyceryl palmitostearate, inhalation lactose, isomalt, kaolin, lactitol, lactose, anhydrous, monohydrate and corn starch, spray dried monohydrate and microcrystalline cellulose, maltodextrin, maltose, mannitol, medium-chain triglycerides, microcrystalline cellulose, polydextrose, polymethacrylates, simethicone, sorbitol, pregelatinized starch, sterilizable maize, sucrose, sugar spheres, sulfobutylether β-cyclodextrin, talc, tragacanth, trehalose, or xylitol. More particular examples of diluents include cellulose powdered, silicified microcrystalline cellulose acetate, compressible sugar, corn starch and pregelatinized starch, dextrose, fructose, glyceryl palmitostearate, anhydrous, monohydrate and corn starch, spray dried monohydrate and microcrystalline cellulose, maltodextrin, maltose, mannitol, medium-chain triglycerides, microcrystalline cellulose, polydextrose, sorbitol, starch, pregelatinized, sucrose, sugar spheres, trehalose, or xylitol.

“Lubricant” refers to materials that prevent or reduce ingredients from clumping together and from sticking to the tablet punches or capsule filling machine. Lubricants also ensure that tablet formation and ejection can occur with low friction between the solid and die wall. Particular examples of lubricants include canola oil, hydrogenated castor oil, cottonseed oil, glyceryl behenate, glyceryl monostearate, glyceryl palmitostearate, medium-chain triglycerides, mineral oil, light mineral oil, octyldodecanol, poloxamer, polyethylene glycol, polyoxyethylene stearates, polyvinyl alcohol, starch, or hydrogenated vegetable oil. More particular examples of diluents include glyceryl behenate, glyceryl monostearate, or hydrogenated vegetable oil.

“Disintegrant” refers to material that dissolve when wet causing the tablet to break apart in the digestive tract, releasing the active ingredients for absorption. They ensure that when the tablet is in contact with water, it rapidly breaks down into smaller fragments, facilitating dissolution. Particular examples of disintegrants include alginic acid, powdered cellulose, chitosan, colloidal silicon dioxide, corn starch and pregelatinized starch, crospovidone, glycine, guar gum, low-substituted hydroxypropyl cellulose, methylcellulose, microcrystalline cellulose, or povidone.

The term “colorant” describes an agent that imparts color to a formulation. Particular examples of colorants include iron oxide, or synthetic organic dyes (US Food and Drug administration, Code of Federal Regulations, Title 21 CFR Part73, Subpart B).

The term “plasticizing agent” or “plasticizer” refers to an agent that is added to promote flexibility of films or coatings. Particular examples of plasticizing agent include polyethylene glycols or propylene glycol.

The term “pigment” in the context of the present invention refers to an insoluble coloring agent.

The term “film-coating agent” or ‘coating agent’ or ‘coating material’ refers to an agent that is used to produce a cosmetic or functional layer on the outer surface of a dosage form. Particular examples of film-coating agent include glucose syrup, maltodextrin, alginates, or carrageenan.

“Glidant” refers to materials that are used to promote powder flow by reducing interparticle friction and cohesion. These are used in combination with lubricants as they have no ability to reduce die wall friction. Particular examples of glidants include powdered cellulose, colloidal silicon dioxide, hydrophobic colloidal silica, silicon dioxide, or talc. More particular examples of glidants include colloidal silicon dioxide, hydrophobic colloidal silica, silicon dioxide, or talc.

“Flavoring agents” refers to material that can be used to mask unpleasant tasting active ingredients and improve the acceptance that the patient will complete a course of medication. Flavorings may be natural (e.g., fruit extract) or artificial. Non limiting examples of flavoring agents include mint, cherry, anise, peach, apricot, licorice, raspberry, or vanilla.

The term “Subject” includes mammals such as humans. The terms “human”, “patient” and “subject” are used interchangeably herein.

“Effective amount” means the amount of a compound of the invention that, when administered to a subject for treating a disease, is sufficient to effect such treatment for the disease. The ‘effective amount’ can vary depending on the compound, the disease and its severity, and the age, weight, etc., of the subject to be treated.

“Preventing” or “prevention” refers to a reduction in risk of acquiring or developing a disease or disorder (i.e., causing at least one of the clinical symptoms of the disease not to develop in a subject that may be exposed to a disease-causing agent, or predisposed to the disease in advance of disease onset).

The term “prophylaxis” is related to “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Treating” or “treatment” of any disease or disorder refers, in one embodiment, to ameliorating the disease or disorder (i.e., arresting the disease or reducing the manifestation, extent or severity of at least one of the clinical symptoms thereof). In another embodiment “reating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron, and 13 Emission Topography (PET) studies for examining substrate receptor occupancy.

All isotopic variants of the compounds provided herein, radioactive or not, are intended to be encompassed within the scope of the invention.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

The term “alkyl” as used herein, whether used alone or as part of another group, refers to a substituted or unsubstituted aliphatic hydrocarbon chain and includes, but is not limited to, straight and branched chains containing from 1 to 20 carbon atoms, preferably from 2 to 20, from 1 to 10, from 2 to 10, from 1 to 8, from 2 to 8, from 1 to 6, from 2 to 6, from 1 to 4, from 2 to 4, from 1 to 3 carbon atoms, unless explicitly specified otherwise. Illustrative alkyl groups can include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

The term “alkenyl” as used herein, whether used alone or as part of another group, refers to a substituted or unsubstituted aliphatic hydrocarbon chain and includes, but is not limited to, straight and branched chains having 2 to 8 carbon atoms and containing at least one carbon-carbon double bond.

The term “alkynyl” as used herein, whether used alone or as part of another group, refers to a substituted or unsubstituted aliphatic hydrocarbon chain and includes, but is not limited to, straight and branched chains having 1 to 6 carbon atoms and containing at least one carbon-carbon triple bond.

The term “alkoxy” as used herein, whether used alone or as part of another group, refers to alkyl-O— wherein alkyl is hereinbefore defined.

The term “cycloalkyl” as used herein, whether used alone or as part of another group, refers to a monocyclic, bicyclic, tricyclic, fused, bridged or spiro monovalent saturated hydrocarbon moiety, wherein the carbon atoms are located inside or outside of the ring system. Any suitable ring position of the cycloalkyl moiety may be covalently linked to the defined chemical structures. Illustrative cycloalkyl groups can include, but are not limited to, cyclopropyl, cyclopropylmethyl, cyclobutyl, cyclopentyl, cyclohexyl, cyclohexylmethyl, cyclohexylethyl, cycloheptyl, norbornyl, adamantly, spiro[4,5]decanyl, and homologs, isomers and the alike.

The term “aryl” as used herein, whether used alone or as part of another group, refers to an aromatic carbocyclic ring system having 6 to 30 carbon atoms, preferably 6 to 10 carbon atoms, optionally substituted with 1 to 3 substituents independently selected from halogen, nitro cyano, hydroxy, alkyl, alkenyl, alkoxy, cycloalkyl, amino, alkylamino, dialkylamino, carboxy, alkoxycarbonyl, haloalkyl, and phenyl.

The term “phenyl” as used herein, whether used alone or as part of another group, refers to a substituted or unsubstituted phenyl group.

The term “heteroaryl” as used herein, whether used alone or as part of another group, refers to a 3 to 30 membered aryl heterocyclic ring, which contains from 1 to 4 heteroatoms selected from the group consisting of O, N, Si, P and S atoms in the ring and may be fused with a carbocyclic or heterocyclic ring at any possible position.

The term “heterocycloalkyl” as used herein, whether used alone or as part of another group, refers to a 5 to 7 membered saturated ring containing carbon atoms and from 1 to 2 heteroatoms selected from the group consisting of O, N and S atoms.

The term “halogen or halo” as used herein, refers to fluoro, chloro, bromo or iodo.

The term “haloalkyl” as used herein, whether used alone or as part of another group, refers to an alkyl as hereinbefore defined, independently substituted with 1 to 3, F, Cl, Br or I.

The term “about” as used herein, refers that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, in phrase “about X to Y,” is the same as “about X to about Y,” that is the term “about” modifies both “X” and “Y.”

The term “compound” as used herein, refers to salts, solvates, complexes, isomers, stereoisomers, diastereoisomers, tautomers, and isotopes of the compound or any combination thereof.

The term “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are used in their inclusive, open-ended, and non-limiting sense.

The term “racemic” as used herein refers to a mixture of the (+) and (−) enantiomers of a compound wherein the (+) and (−) enantiomers are present in approximately a 1:1 ratio. The terms “substantially optically pure,” “optically pure,” and “optically pure enantiomers,” as used herein, mean that the composition contains greater than about 90% of a single stereoisomer by weight, preferably greater than about 95% of the desired enantiomer by weight, and more preferably greater than about 99% of the desired enantiomer by weight, based upon the total weight.

The term “enantiomer” refers to a stereoisomer that is a non-superimposable mirror image of each other. A diastereomer is a stereoisomer with two or more stereocenters, and the isomers are not mirror images of each other.

The term “triggering receptor expressed on myeloid cells” or “TREM” as used herein, refers to a group of activating receptors which are selectively expressed on different types of myeloid cells, such as mast cells, monocytes, macrophages, dendritic cells (DCs), and neutrophils, and may have a predominant role in immune and inflammatory responses. TREMs are primarily transmembrane glycoproteins with a Ig-type fold in their extracellular domain and, bence, belong to the Ig-SF. These receptors contain a short intracellular domain, but lack docking motifs for signaling mediators and require adapter proteins, such as DAP12, for cell activation.

The term “myeloid cells” as used herein, refers to a series of bone marrow-derived cell lineages including granulocytes (neutrophils, eosinophils, and basophils), monocytes, macrophages, and mast cells. Furthermore, peripheral blood dendritic cells of myeloid origin, and dendritic cells and macrophages derived in vitro from monocytes in the presence of appropriate culture conditions, are also included.

Triggering via TREM-1 results in the production of proinflammatory cytokines, chemokines and reactive oxygen species, and leads to rapid degranulation of neutrophilic granules, and phagocytosis. Since interfering with TREM-1 engagement leads to the simultaneous reduction in production and secretion of a variety of proinflammatory mediators, TREM-1 represents an attractive target for treating chronic inflammatory disorders. Indeed, a role for TREM-1 has been demonstrated in a variety of inflammatory disorders, including, but not limited to, acute and chronic inflammatory disorders, sepsis, acute endotoxemia, encephalitis, Chronic Obstructive Pulmonary Disease (COPD), allergic inflammatory disorders, asthma, pulmonary fibrosis, pneumonia, Community acquired pneumonia (CAP), Ventilator associated pneumonia (VAP), Acute respiratory infection, Acute respiratory distress syndrome (ARDS), Infectious lung diseases, Pleural effusion. Peptic ulcer, Helicobacter pylori infection, hepatie granulomatosis, arthritis, rheumatoid arthritis, osteoarthritis, inflammatory osteolysis, ulcerative colitis, psoriasis, vasculitis, autoimmune disorders, thyroiditis, Meliodosis, (mesenteric) Ischemia reperfusion, Filovirus infection, Infection of the urinary tract. Bacterial meningitis, Salmonella enterica infection, Marburg and Ebola viruses infections, and in particular Inflammatory Bowel Disease (IBD).

Inflammatory bowel disease (IBD) covers a group of disorders in which the intestines become inflamed (red and swollen), probably as a result of an immune reaction of the body against its own intestinal tissue. Two major types of IBD are described: ulcerative colitis (UC) and Crohn's disease (CD). Ulcerative colitis is limited to the colon (large intestine). Crohn's disease can involve any part of the gastrointestinal tract from the mouth to the anus, but it most commonly affects the small intestine and/or the colon. Both ulcerative colitis and Crohn's disease vary in intensity and severity during the course of the disease. When there is severe inflammation, the disease is considered to be in an active stage and the person experiences a flare-up of the condition. When the degree of inflammation is less (or absent), the person usually is without symptoms, and the disease is considered to be in remission. In IBD, factor or factors trigger the body's immune system to produce an inflammatory reaction in the intestinal tract that continues without control. As a result of the inflammatory reaction, the intestinal wall is damaged leading to bloody diarrhea and abdominal pain amongst other signs.

It will be appreciated that compounds of the invention may be metabolized to yield biologically active metabolites.

### II. Inhibitors of TREM-1

### A. Genus Structure Based on VJDT8293 (Formula I)

One embodiment provides a compound of Formula I:

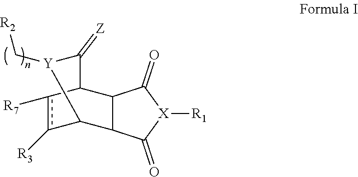

wherein=
[symbol] is a single or a double bond;
X and Y are each independently N or CH;
Z is independently CH₂, O, S, NH;
R₁ is independently alkyl, alkenyl, alkoxy, cycloalkyl, aryl, heteroaryl, wherein each of which is unsubstituted or substituted with H, OH, halogen, —COOH, —COOR₄, CH₂OH, —CH₂OR₄, —COSR₄, —CONH₂, —CH₂NH₂, CH₂NHR₅, —CH₂N₅R₆, —NCO, —CH₂-halogen, —CHO, —CN, —CONH—CO—R₄, —CH₂O—CO—O—R₄; NO₂, NH₂, NHR₅, NR₅R₆;
R₂ is independently alkyl, alkenyl, alkoxy, cycloalkyl, aryl, heteroaryl, wherein each of which is unsubstituted or substituted with H, OH, halogen, —COOH, —COOR₄, CH₂OH, —CH₂OR₄, —COSR₄, —CONH₂, —CH₂NH₂, CH₂NHR₅, —CH₂N₅R₆, —NCO, —CH₂-halogen, —CHO, —CN, —CONH—CO—R₄, —CH₂O—CO—O—R₄; NO₂, NH₂, NHR₅, NR₅R₆;
R₃ is independently H, OH, halogen, —COOH, —COOR₄, CH₂OH, —CH₂OR₄, —COSR₄, —CONH₂, —CH₂NH₂, CH₂NHR₅, —CH₂N₅R₆, —NCO, —CH₂-halogen, —CHO, —CN, —CONH—CO—R₄, —CH₂O—CO—O—R₄; NO₂, NH₂, NHR₅, NR₅R₆; and
R₄ is independently H, halogen, alkyl, aryl, heteroaryl;
R₅ and R₆ are independently H, halogen, alkyl, aryl, heteroaryl;
R₇ is independently H, CN, halogen, alkyl, alkoxy, aryl, heteroaryl; and
n is 1, 2, 3, 4, 5 or 6;
or an enantiomer, tautomer, isomer, exo and endo stereoisomers, bioisosteres, hydrate, solvate, racemate, deuterated analogs, zwitterion, polymorph, prodrug, or a pharmaceutically acceptable salt thereof.

In one embodiment, R₁ is unsubstituted or substituted aryl.

In other embodiment, R₂ is unsubstituted or substituted aryl.

In some embodiment, R₃ is H or CN.

In further embodiment, R₇ is H, CN or OCH₃.

In one embodiment, X is N.

In other embodiment, Y is N.

In another embodiment, Z is O.

### B. VJDT8293 (Formula II)

Another embodiment provides a compound of Formula II:

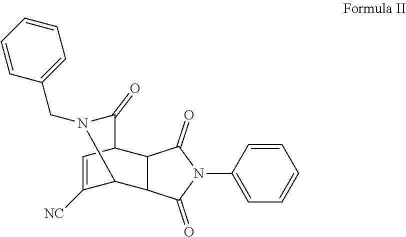 [SMILES: N#CC1=CC2C(=O)N(Cc3ccccc3)C1C1C(=O)N(c3ccccc3)C(=O)C21]

or an enantiomer, tautomer, isomer, exo and endo stereoisomers, bioisosteres, hydrate, solvate, racemate, deuterated analogs, zwitterion, polymorph, prodrug, or a pharmaceutically acceptable salt thereof.

### III. Pharmaceutical Formulations

The compounds of Formulas I and II and mixtures thereof can be formulated into a pharmaceutical composition. Pharmaceutical compositions can be administered by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or by using bioerodible inserts and can be formulated in dosage forms appropriate for each route of administration. The compositions can be administered systemically.

The compounds of Formulas I and II can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g., as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or by promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” consists of all components present in the pharmaceutical formulation other than the active ingredient or ingredients. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical Dosage Form Tablets”, eds. Liberman et. al. (New York, Marcel Dekker, Inc., 1989), “Remington—The Science and Practice of Pharmacy”, 20th ed., Lippincott Williams & Wilkins, Baltimore, MD, 2000, and “Pharmaceutical Dosage Forms and Drug Delivery Systems”, 6^{th} Edition, Ansel et. al., (Media, PA: Williams and Wilkins, 1995) which provides information on carriers, materials, equipment and process for preparing tablets, capsules, and delayed release dosage forms of tablets, capsules, and granules.

The compounds of Formulas I and II can be administered to a subject with or without the aid of a delivery vehicle. Appropriate delivery vehicles for the compounds are known in the art and can be selected to suit the particular active agent. For example, in some embodiments, the active agent(s) is/are incorporated into or encapsulated by, or bound to, a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly (lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

### A. Formulations for Parenteral Administration

Compounds of Formulas I and II and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents like sterile water and buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### B. Oral Immediate Release Formulations

Suitable oral dosage forms of the compounds of Formulas I and II include tablets, capsules, solutions, suspensions, syrups, and lozenges. Tablets can be made using compression or molding techniques well known in the art. Gelatin or non-gelatin capsules can be prepared as hard or soft capsule shells which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, polysaccharides and the like.

Additionally, the coating material may contain conventional carriers such as plasticizers, pigments, colorants, glidants, stabilization agents, pore formers and surfactants, and the like.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar and the like.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid polyvinylpyrrolidone, and the like.

Lubricants are used to facilitate tablet manufacture. Examples of suitable lubricants include, but are not limited to, magnesium stearate, calcium stearate, stearic acid, glycerol behenate, polyethylene glycol, talc, mineral oil, and the like.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp), and the like.

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide and the like. Examples of amphoteric surfactants include sodium N-dodecyl-.beta.-alanine, sodium N-lauryl-.beta.-iminodipropionate, myristoamphoacetate, lauryl betaine, lauryl sulfobetaine and the like.

If desired, the tablets, beads granules or particles may also contain minor amount of nontoxic auxiliary substances such as wetting or emulsifying agents, dyes, pH buffering agents, and preservatives.

### C. Extended Release Dosage Forms

The extended release formulations of compounds of Formulas I and II are generally prepared as diffusion or osmotic systems, for example, as described in “Remington—The Science and Practice of Pharmacy” (20th ed., Lippincott Williams & Wilkins, Baltimore, MD, 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, polyethylene, and the like. Hydrophilic polymers include, but are not limited to, methylcellulose, hydroxypropylcellulose, hydroxypropylmethylcellulose, sodium carboxymethylcellulose, and carbopol 934, polyethylene oxides and the like. Fatty compounds include, but are not limited to, various waxes such as carnauba wax, glyceryl tristearate and the like.

Alternatively, extended release formulations of the compounds of Formulas I and II can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride powdered sugar and the like. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### D. Delayed Release Dosage Forms

In some embodiments delayed release formulations of compounds of formulas I and II are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads, and granules prepared with different quantities of various coating materials. It is the combination of materials, method and form of application that produce the desired release characteristics, which one can determine without undue experimentation from clinical studies.

The coating composition may include conventional additives, such as plasticizers, pigments, colorants, stabilizing agents, glidants, and the like. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, acetylated monoglycerides and the like. A stabilizing agent is preferably used to stabilize particles in the dispersion. Typical stabilizing agents are nonionic emulsifiers such as sorbitan esters, polysorbates, polyvinylpyrrolidone and the like. Glidants are recommended to reduce sticking effects during film formation and drying, and will generally represent approximately 25 wt. % to 100 wt. % of the polymer weight in the coating solution. One effective glidant is talc. Other glidants such as magnesium stearate, glycerol monostearates and the like may also be used. Pigments such as titanium dioxide may also be used. Small quantities of an anti-foaming agent, such as a silicone (for example, simethicone), may also be added to the coating composition.

### E. Formulations for Mucosal and Pulmonary Administration

The compounds of Formulas I and II and compositions thereof can also be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the compounds are formulated for pulmonary delivery such as by intranasal administration or oral inhalation. The respiratory tract is the structure involved in the exchange of gases between the atmosphere and the blood stream. The lungs are branching structures ultimately ending with alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Another embodiment of the present invention provides for nasal delivery for administration of the compounds of Formulas I and II. The compounds of Formulas I and II can be formulated as an aerosol. The term aerosol refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and those for administration as solutions. Aerosols for the delivery of therapeutic agents to the respiratory tract are known in the art. For administration via the upper respiratory tract, the formulation can be formulated into a solution, for example, water or isotonic saline, buffered or un-buffered, or as a suspension, for intranasal administration as drops or as a spray. Preferably, such solutions or suspensions are isotonic relative to nasal secretions and of about the same pH, ranging from about pH 4.0 to about pH 7.4 or from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS), and the like. Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation and should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+nebulizer (PARI Respiratory Equipment, Monterey, CA).

Dry powder formulations (“DPFs”) with large particle size have improved flowability characteristics, such as less aggregation, easier aerosolization, and are subject potentially to less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

### F. Topical and Transdermal Formulations

Transdermal formulations containing the compounds of Formulas I and II may also be prepared. These will typically be gels, ointments, lotions, sprays, or patches, all of which can be prepared using standard technology. Transdermal formulations can include penetration enhancers.

An “oil” is a composition containing at least 95% wt of a lipophilic substance. Examples of lipophilic substances include, but are not limited to, naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are externally applied agents that softens or soothes skin and are generally known in the art and listed in compendia, such as the “Handbook of Pharmaceutical Excipients”, 4th Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophillic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers will buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, thimerosal and the like.

Additional agents that can be added to the formulation include penetration enhancers. In some embodiments, the penetration enhancer increases the solubility of the drug, improves transdermal delivery of the drug across the skin, in particular across the stratum corneum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly (l0 oxyethylene ether), BRIJ® 78 (stearyl poly (20) oxyethylene ether), BRIJ® 96 (oleyl poly (l0) oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8 (2): 173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, and the like, include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

In some embodiments, the composition is formulated for transdermal delivery and administered using a transdermal patch. In some embodiments, the formulation, the patch, or both are designed for extended release of the curcumin conjugate.

Exemplary symptoms, pharmacologic, and physiologic effects are discussed in more detail below.

### G. Methods of Manufacture

As will be appreciated by those skilled in the art and as described in the pertinent texts and literature, a number of methods are available for preparing formulations containing the compounds of Formulas I and II including but not limited to tablets, beads, granules, microparticle, or nanparticles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, for example, by using a conventional coating pan, an airless spray technique, or fluidized bed coating equipment (with or without a Wurster insert). For detailed information concerning materials, equipment, and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

An exemplary method for preparing extended release tablets includes compressing a drug-containing blend, e.g., blend of drug-containing granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding technology. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, and the like, extruding the blend, spheronizing the extrudate, drying, and optionally coating to form the immediate release beads.

### IV. Methods of Use

The compounds of Formulas I and II and pharmaceutical compositions thereof are useful for the treatment of TREM-related disease, disorder or condition. In some embodiments, the TREM-related disease, disorder or condition is inflammatory disease, cardiovascular disease, and cancer. In other embodiments, the present invention provides methods of inhibiting TREM-1 comprising contacting the TREM-1 cells with a compound of Formula I or II and pharmaceutical composition thereof.

### A. Inflammatory Diseases

In one embodiment, the present invention provides methods of treating an inflammatory disease, disorder or condition in a subject in need thereof comprising administering to the subject a therapeutically effective amount of the compound or a pharmaceutical composition thereof.

Representative inflammatory diseases, disorders or conditions that can be inhibited or treated by the compound of formula I or pharmaceutical composition thereof includes, but are not limited to, acute and chronic inflammatory disorders, sepsis, acute endotoxemia, encephalitis, Chronic Obstructive Pulmonary Disease (COPD), allergic inflammatory disorders, asthma, pulmonary fibrosis, pneumonia, Community acquired pneumonia (CAP). Ventilator associated pneumonia (VAP), Acute respiratory infection, Acute respiratory distress syndrome (ARDS), Infectious lung diseases, Pleural effusion, Peptic ulcer, Helicobacter pylori infection, hepatic granulomatosis, arthritis, rheumatoid arthritis, osteoarthritis, inflammatory osteolysis, ulcerative colitis, psoriasis, vasculitis, autoimmune disorders, thyroiditis, Meliodosis, (mesenteric) Ischemia reperfusion, Filovirus infection, Infection of the urinary tract, Bacterial meningitis, Salmonella enterica infection, Marburg and Ebola viruses infections, and in particular Inflammatory Bowel Disease (IBD).

### B. Cardiovascular Diseases

In other embodiments of the present invention, methods are provided for treating a cardiovascular disease, disorder, or condition in a subject in need thereof comprising administering to the subject a therapeutically effective amount of the compound or a pharmaceutical composition thereof.

Representative cardiovascular diseases, disorders, or conditions that can be inhibited or treated by the compound of formula I or pharmaceutical composition thereof include, but are not limited to, atherosclerosis, arteriosclerosis, reperfusion/ischemia in stroke, cardiac hypertrophy, respiratory diseases, heart attacks, and myocardial ishemia.

### C. Cancers

In some embodiments, the present invention provides methods of treating a cancer in a subject in need thereof comprising administering to the subject a therapeutically effective amount of the compound or a pharmaceutical composition thereof.

Representative cancers that can be inhibited or treated by the compound of formula I or pharmaceutical composition thereof include, but are not limited to, squamous cell carcinoma, small-cell lung cancer, non-small cell lung cancer (NSCLC), lung adenocarcinoma, squamous cell lung cancer, peritoneum cancer, hepatocellular cancer, stomach cancer, gastrointestinal cancer, esophageal cancer, pancreatic cancer, glioblastoma, cervical cancer, ovarian cancer, liver cancer, bladder cancer, breast cancer, colon cancer, rectal cancer, colorectal cancer, endometrial cancer, uterine cancer, salivary gland carcinoma, melanoma, renal cancer, prostate cancer, vulval cancer, thyroid cancer, hepatocellular carcinoma (HCC), anal carcinoma, penile carcinoma, head and neck cancer and the like.

### EXAMPLES

### Example 1: Receptor-Based Virtual Screening

### Methods and Materials

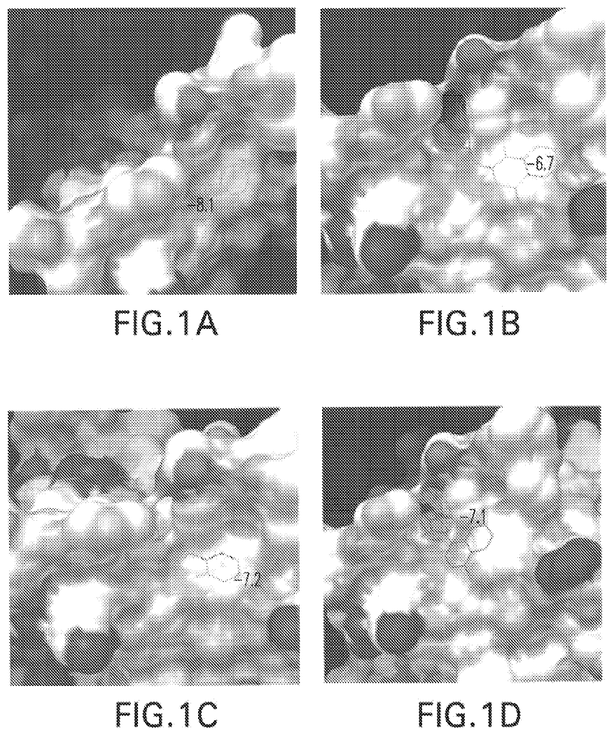

To design novel TREM-1 inhibitors, receptor-based virtual screening (RBVS) was performed against the NCI diversity database. RBVS, a high throughput computational drug discovery approach, has become a potentially powerful and inexpensive method for searching novel lead compounds in drug development. RBVS is based on a molecular docking technique requiring knowledge of the three-dimensional structure of the target protein binding site and its likelihood to bind to proteins. A total of 1,593 compounds from the NCI diversity set with nonredundant structure to inhibit human TREM-1 were analyzed. Using different docking algorithms, combined with subsequent post-docking analyses, 154 candidate compounds with high scoring functions that all bind to the ligand binding site of the extracellular domain of human TREM-1 were found (FIGS. 1A-1D).

### Results

Representative data from 154 candidate compounds with higher binding mode to TREM-1 are shown in FIGS. 1A-1D.

### Example 2: Chemical Synthesis

Compounds of formula I and II were synthesized.

### Synthesis of VJDT 8293 (3aR,7aR)-9-benzyl-1,3,8-trioxo-2-phenyl-2,3,3a,4,7,7a-hexahydro-1H-4,7-(epiminomethano) isoindole-5-carbonitrile)

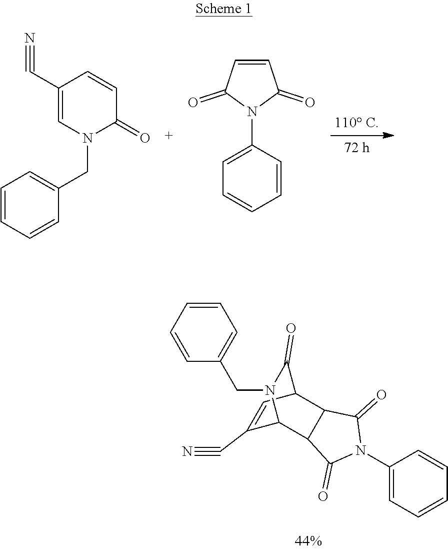 [SMILES: N#Cc1ccc(=O)n(Cc2ccccc2)c1 ; O=C1C=CC(=O)N1c1ccccc1 ; N#CC1=CC2C(=O)N(Cc3ccccc3)C1C1C(=O)N(c3ccccc3)C(=O)C21]

### Methods and Materials

VJDT 8293 was synthesized using Diels-Alders reaction, which was carried out via 1:1 molar condensation of a diene and a dienophile yielding 44% of VJDT 8293 ((3aR,7aR)-9-benzyl-1,3,8-trioxo-2-phenyl-2,3,3a,4,7,7a-hexahydro-1H-4,7-(epiminomethano) isoindole-5-carbonitrile) as shown in Scheme 1.

### Example 3: Inhibition of TREM-1

### Methods and Materials

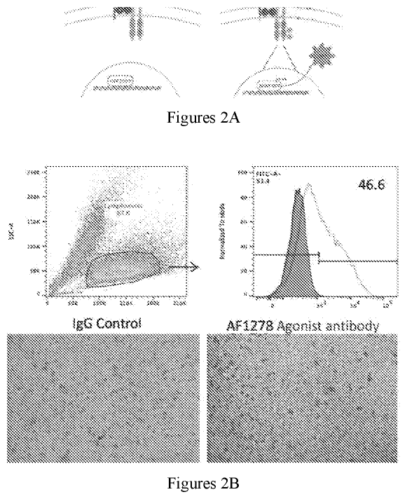

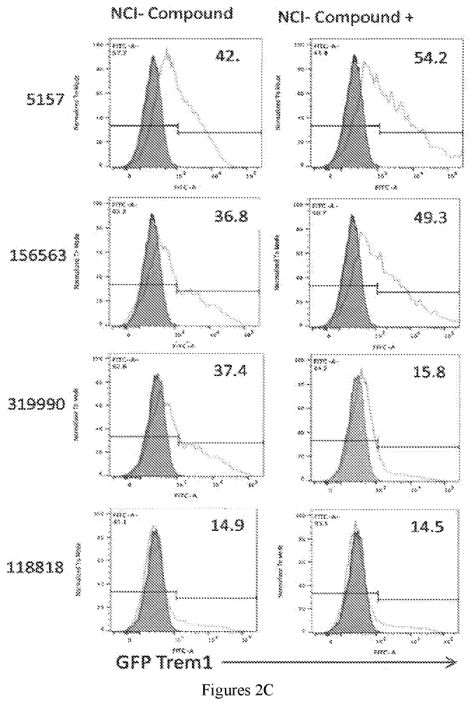

For blocking studies, TREM-1-positive cells were preincubated with each tested compounds for 4 hrs followed by stimulation with agonist antibody AF1278 for an additional 24 hrs (FIGS. 2A-2B). As seen in FIG. 2C, several compounds, namely compound 5157, compound 156563, compound 319990 and compound 118818 (9-Benzyl-1,3-dioxo-2-phenyl-2,3,3a,4,7,7a-hexahydro-1H-4,7-(epiminomethano) isoindole-5-carbonitrile), bind TREM-1 receptor on reporter cells and activate the receptor as determined by significant enhancement of GFP expression. Moreover, these compounds did not provide any inhibitory effect on agonist-activated TREM-1-specific cells.

### Results

It was determined that Compounds 319990 and 118818 (9-Benzyl-1,3-dioxo-2-phenyl-2,3,3a,4,7,7a-hexahydro-1H-4,7-(epiminomethano) isoindole-5-carbonitrile) have a strong TREM-1 inhibitory effect. However, Compound 319990 demonstrates a higher cytotoxicity effect on reporter cells compared to Compound 118818 (FIG. 2C).

### Example 4: Assessment of Cytotoxicity

### Methods and Materials

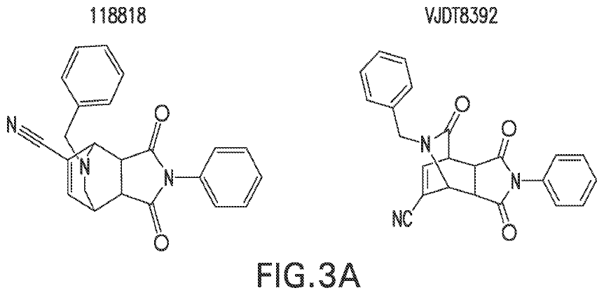

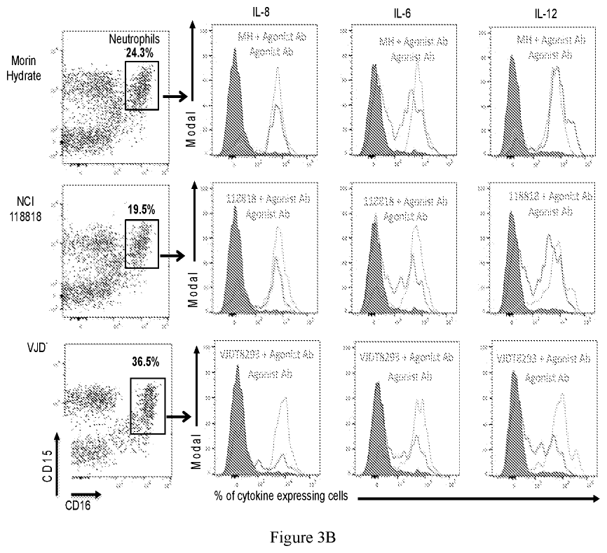

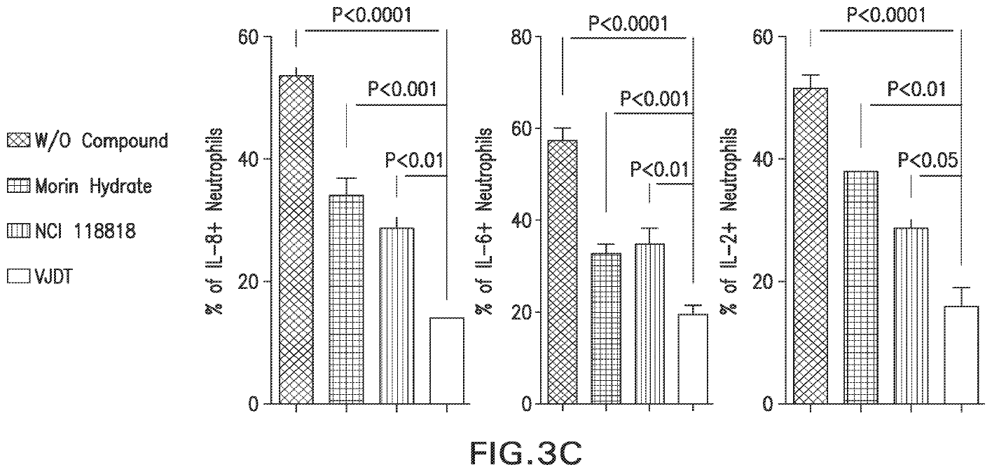

Human cells were treated with Morin Hydrate (MH), NCI 118818 (9-Benzyl-1,3-dioxo-2-phenyl-2,3,3a,4,7,7a-hexahydro-1H-4,7-(epiminomethano) isoindole-5-carbonitrile), or VJDT8293, and the effects were analyzed on proinflammatory cytokine production (protein level) (FIG. 3B). Peripheral blood mononuclear cells (PBMCs) were isolated from healthy volunteers and were stimulated with TREM-1 agonist Ab for 24 hrs, at concentration of 4 μg/ml (red) or pretreated with indicated compounds (at 50 μM) for 2 hrs and stimulated with agonist AB at concentration of 4 pg/mlfor 24 hrs (blue)

### Results

FIG. 3B demonstrates that the number of neutrophils was decreased in experiments using Morin Hydrate (MH) and NCI 118818 treatment (24.3%, and 19,5%, respectively) indicating the level of cytotoxicity of the compounds. However, the cells treated with Compound JDT 8392 demonstrate minimal cytotoxicity (36.5%) and, therefore, was selected as the best compound compared to MH or 118818.

### Example 5: Effects on Pro-Inflammatory Cytokines

### Methods and Materials

Quantification (percentage) of cytokine-positive neutrophils from untreated, Morin Hydrate (MH)-treated, 118818-treated, and VJDT 8293-treated compounds (from triplicates) was performed. Cells isolated from the same healthy volunteer have been used for indicated experiments.

### Results

The data shows that the VIDT 8293 compound provides a much stronger inhibitory effect on TREM-1 mediated pro-inflammatory cytokine production by activated human neutrophils with less cytotoxicity. FIG. 3C shows a bar graph of flow cytometry analyses comparing untreated cells with those treated by compound MH (Morin Hydrate), compound NCI 118818 and compound VJDT8293. PBMCs were stained cell surface markers CD15 and CD16. In addition, intracellular staining for proinflammatory cytokines, such as IL-8, IL-6 and IL-12, was performed. PBMCs were gated on CD15, CD16 markers to identify the population of neutrophils.

### Example 6: VJDT 8293 Treatment Significantly Attenuates Inflammatory Cell Infiltration at Early Stages of Liver Fibrosis

### Methods and Materials

TABLE 1
Treatment with VJDT

| | VJDT: 25 mg/kg oral | | | | | | |
|---|---|---|---|---|---|---|---|
| Mouse | | | | | CCL: | | |
| Treatment | | | | | 2 ml/kg | | |
| Days | 1 | 2 | 3 | 4 | 5 | 6 | Sacrifice |
| | | | | | | | and |
| | | | | | | | collect |

Wild-type mice (Trem1^{+/+}) were orally administered VJDT (25 mg/kg) 5 consecutive days prior to injury with CCl4 (2 ml/kg, diluted 1:4 in corn oil) (24 hrs) or one dosage of control corn oil (Sigma-Aldrich, St. Louis, MO, USA). Liver cells were isolated 24 hrs after CCl4 injury, perfused with calcium and magnesium-free 40° C. prewarmed HBSS solution (Corning Life Sciences, Corning, NY, USA) and digested with collagenase type I. The cell suspension was filtered through a 70 μm cell strainer and then centrifuged at 50 g for 5 min at 4° C. to eliminate hepatocytes. Cells remaining in the supernatant were layered on HISTOPAQUE®-1083 and centrifuged at 1000 g for 15 min at 4° C. without a break. The non-parenchymal cell fraction was washed once with PBS at 300 g for 10 min at 4° C., followed by flow cytometry.

### Results

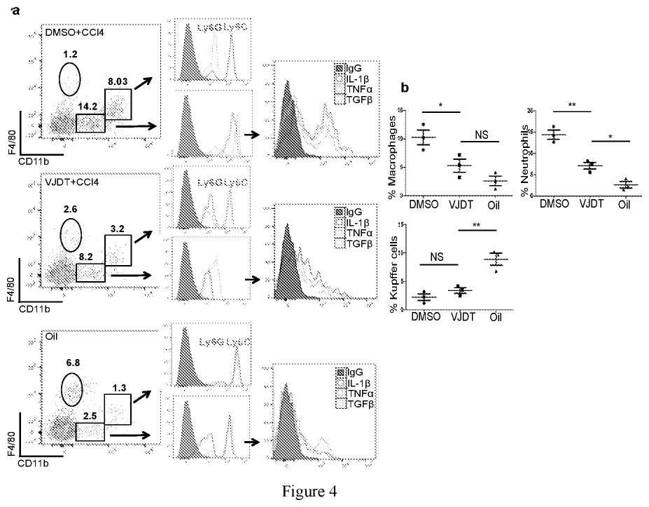

FIG. 4 shows VIDT 8293 treatment significantly attenuates inflammatory cell infiltration at early stages of liver fibrosis. Nonparenchymal cells isolated from Trem1^{+/+} livers with CCl4 injury receiving VJDT treatment were analyzed by flow cytometry to evaluate the infiltration of myeloid cells. Liver-associated cells were classified into 3 populations: F4/80⁺CD11b⁻ resident Kupffer cells, F4/80⁺CD11b⁺Ly6C^{hi}Ly6G^{lo} monocyte-derived macrophages and F4/80⁺CD11b⁺Ly6C^{lo}Ly6G^{hi} inflammatory neutrophils. FIG. 4a shows control group receiving DMSO vehicle showed significant infiltration of macrophages (10-15%) and inflammatory neutrophils (15-22%) producing IL-1B, TNF, and TGF-β1 due to CCl4 injury. In contrast, FIG. 4b shows VIDT treatment had strong protective effect to liver injury as demonstrated by significantly reduced infiltration of both populations: macrophages (3-6%, p<0.001), and neutrophils (8-10%, p<0.01). Moreover, VJDT treatment additionally significantly decreased cytokine secretion in neutrophils as compared to the control group. Control mice with no CC14 injury showed minimal level of infiltrating cells and a normal population of resident Kupffer cells (10-15%).

### Example 7: VJDT 8293 Treatment Inhibits Tumor Cell Proliferation, Migration and Plasticity

### Methods and Materials

B16F10 cells were cultured in 6 well plates incubated with either vehicle alone or vehicle with VJDT at 50 μM and streaks were made using a tip when cells were at 80% confluency. Streaks were photographed at 0 hrs or 24 hrs at 100×. B16F10 cells were treated with VJDT at 50 μM concentration followed by 10 μM CFSE labeling according to manufacturer's protocol. After 48 hrs cells were analyzed by flow cytometry for CFSE intensity that will be inversely proportional to cell proliferation. Further, cell cycle assay via propidium iodide on B16F10 cells with or without VIDT treatment was performed for investigating the anti-proliferative effect of VJDT in tumor cells

### Results

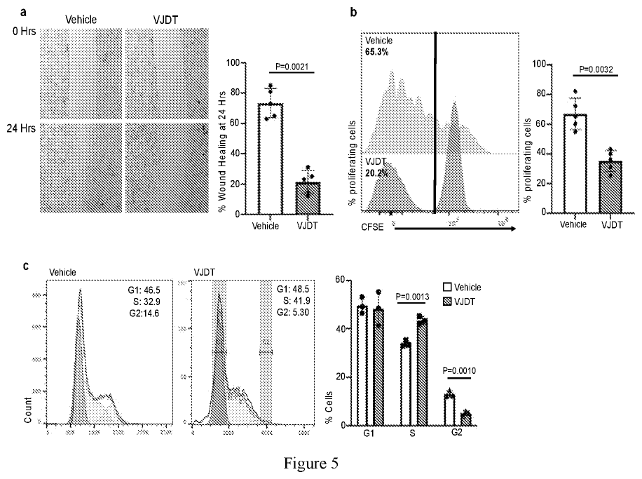

FIG. 5 shows VIDT 8293 treatment inhibits tumor cell proliferation, migration and plasticity. FIG. 5A shows VIDT treatment significantly inhibited B16F10 murine melanoma cell migration in wound healing assay. FIG. 5B shows VIDT treatment significantly inhibited cell proliferation. FIG. 5C shows VJDT treatment produces significant cell cycle arrest in the S and G2 phases of cellular division. In vitro studies provide substantial data supporting the anti-cancer effects of VIDT treatment affecting tumor cell proliferation, migration and plasticity. Overall, there is compelling evidence for VIDT to be an effective therapeutic drug specifically targeting the novel TREMI pathways during tumor initiation, promotion, and progression.

### Example 8: Determination of VJDT 8293 Half Maximal Inhibitory Concentration (IC₅₀) to Inhibit Cell Proliferation of Human Hepatocellular Carcinoma Cells (HepG2) In Vitro

### Methods and Materials

Cell proliferation was measured using Cell Counting Kit-8 (CCK-8, GLPBIO, Montclair, CA. Catalog No. GK10001) according to the manufacturer's instructions. HepG2 cells were seeded in a 96-well plate at 3×10³ cells/well and incubated at 37° C. in complete medium for 24 hrs before being treated with the indicated concentrations of VIDT for 72 hrs. After treatment, cells were incubated with 10 μl CCK8 at 37° C. for 2 hrs. Absorbance at 450 nm was determined using a microplate reader. The IC₅₀ value was calculated on the nonlinear regression fit method by the GraphPad Prism software. Results (n=6) are displayed as the mean±SD.

### Results

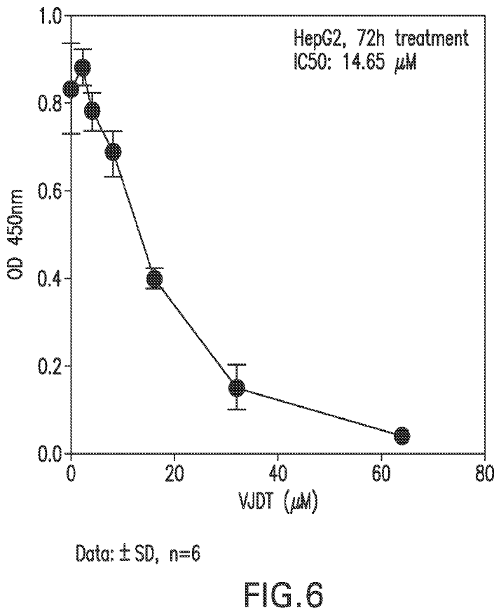

FIG. 6 shows IC₅₀ of VJDT 8293 as 14.65 μM to inhibit cell proliferation of human hepatocellular carcinoma cells (HepG2) in vitro after 72 hrs treatment.

While in the foregoing specification this invention has been described in relation to certain embodiments thereof, and many details have been put forth for the purpose of illustration, it will be apparent to those skilled in the art that the invention includes additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A compound of Formula I: 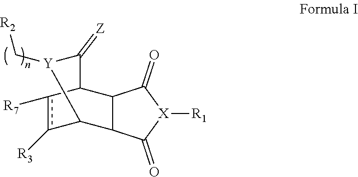
wherein [symbol]
is a single or a double bond;
X and Y are each independently N or CH;
Z is independently CH₂, O, S, NH;
R₁ is independently alkyl, alkenyl, alkoxy, cycloalkyl, aryl, heteroaryl, wherein each of which is unsubstituted or substituted with H, OH, halogen, —COOH, —COOR₄, CH₂OH, —CH₂OR₄, —COSR₄, —CONH₂, —CH₂NH₂, CH₂NHR₅, —CH₂N₅R₆, —NCO, —CH₂-halogen, —CHO, —CN, —CONH—CO—R₄, —CH₂O—CO—O—R₄; NO₂, NH₂, NHR₅, NR₅R₆;
R₂ is independently alkyl, alkenyl, alkoxy, cycloalkyl, aryl, heteroaryl, wherein each of which is unsubstituted or substituted with H, OH, halogen, —COOH, —COOR₄, CH₂OH, —CH₂OR₄, —COSR₄, —CONH₂, —CH₂NH₂, CH₂NHR₅, —CH₂N₅R₆, —NCO, —CH₂-halogen, —CHO, —CN, —CONH—CO—R₄, —CH₂O—CO—O—R₄; NO₂, NH₂, NHR₅, NR₅R₆;
R₃ is independently H, OH, halogen, —COOH, —COOR₄, CH₂OH, —CH₂OR₄, —COSR₄, —CONH₂, —CH₂NH₂, CH₂NHR₅, —CH₂N₅R₆, —NCO, —CH₂-halogen, —CHO, —CN, —CONH—CO—R₄, —CH₂O—CO—O—R₄; NO₂, NH₂, NHR₅, NR₅R₆; and
R₄ is independently H, halogen, alkyl, aryl, heteroaryl;
R₅ and R₆ are independently H, halogen, alkyl, aryl, heteroaryl;
R₇ is independently H, CN, halogen, alkyl, alkoxy, aryl, heteroaryl; and
n is 1, 2, 3, 4, 5 or 6;
or an enantiomer, tautomer, isomer, exo and endo stereoisomers, bioisosteres, hydrate, solvate, racemate, deuterated analogs, zwitterion, polymorph, prodrug, or a pharmaceutically acceptable salt thereof.

2. A compound of Formula II: 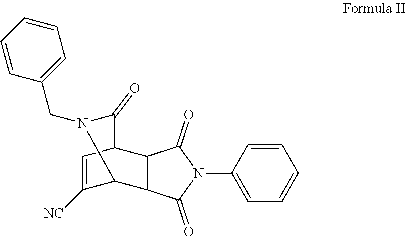 [SMILES: N#CC1=CC2C(=O)N(Cc3ccccc3)C1C1C(=O)N(c3ccccc3)C(=O)C21]
or an enantiomer, tautomer, isomer, exo and endo stereoisomers, bioisosteres, hydrate, solvate, racemate, deuterated analogs, zwitterion, polymorph, prodrug, or a pharmaceutically acceptable salt thereof.

3. The compound of claim 1, wherein the R₁ is unsubstituted or substituted aryl.

4. The compound of claim 1, wherein the R₂ is unsubstituted or substituted aryl.

5. The compound of claim 1, wherein the R₃ is H or CN.

6. The compound of claim 1, wherein the R₇ is H, CN or OCH₃.

7. The compound of claim 1, wherein the X is N.

8. The compound of claim 1, wherein the Y is N.

9. The compound of claim 1, wherein the Z is O.

10. A pharmaceutical composition comprising a compound of claim 1 or a pharmaceutically acceptable salt thereof, and a pharmaceutically acceptable carrier, adjuvant or vehicle.

11. A method of inhibiting TREM-1, the method comprising contacting the TREM-1 cells with a compound of claim 1 or a pharmaceutical composition thereof.

12. A method treating a TREM-related disease, disorder or condition in a subject in need thereof comprising administering to the subject a therapeutically effective amount of the compound of claim 1 or a pharmaceutical composition thereof.

13. The method of claim 12, wherein the TREM-related disease, disorder or condition is inflammatory disease, cardiovascular disease, and cancer.

14. A method treating an inflammatory disease, disorder or condition in a subject in need thereof comprising administering to the subject a therapeutically effective amount of the compound of claim 1 or a pharmaceutical composition thereof.

15. The method of claim 14, wherein the inflammatory disease is selected from acute and chronic inflammatory disorders, sepsis, acute endotoxemia, encephalitis, Chronic Obstructive Pulmonary Disease (COPD), allergic inflammatory disorders, asthma, pulmonary fibrosis, pneumonia, Community acquired pneumonia (CAP), Ventilator associated pneumonia (VAP), Acute respiratory infection, Acute respiratory distress syndrome (ARDS), Infectious lung diseases, Pleural effusion, Peptic ulcer, Helicobacter pylori infection, hepatic granulomatosis, arthritis, rheumatoid arthritis, osteoarthritis, inflammatory osteolysis, ulcerative colitis, psoriasis, vasculitis, autoimmune disorders, thyroiditis, Meliodosis, (mesenteric) Ischemia reperfusion, Filovirus infection, Infection of the urinary tract, Bacterial meningitis, Salmonella enterica infection, Marburg and Ebola viruses infections, and in particular Inflammatory Bowel Disease (IBD).

16. A method treating a cardiovascular disease, disorder or condition in a subject in need thereof comprising administering to the subject a therapeutically effective amount of the compound of claim 1 or a pharmaceutical composition thereof.

17. The method of claim 16, wherein the cardiovascular disease is selected from atherosclerosis, arteriosclerosis, reperfusion/ischemia in stroke, cardiac hypertrophy, respiratory diseases, heart attacks, and myocardial ishemia.

18. A method treating a cancer in a subject in need thereof comprising administering to the subject a therapeutically effective amount of the compound of claim 1 or a pharmaceutical composition thereof.

19. The method of claim 18, wherein the cancer is selected from squamous cell carcinoma, small-cell lung cancer, non-small cell lung cancer (NSCLC), lung adenocarcinoma, squamous cell lung cancer, peritoneum cancer, hepatocellular cancer, stomach cancer, gastrointestinal cancer, esophageal cancer, pancreatic cancer, glioblastoma, cervical cancer, ovarian cancer, liver cancer, bladder cancer, breast cancer, colon cancer, rectal cancer, colorectal cancer, endometrial cancer, uterine cancer, salivary gland carcinoma, melanoma, renal cancer, prostate cancer, vulval cancer, thyroid cancer, hepatocellular carcinoma (HCC), anal carcinoma, penile carcinoma, or head and neck cancer.

20. The method of claim 19, wherein the cancer is melanoma and liver cancer.
